(19) United States
(12) Patent Application Publication
(10) Pub. No.: US 2022/0020228 A1
(43) Pub. Date: Jan. 20, 2022

(54) SYSTEMS AND METHODS FOR MODIFYING GEOMETRY SURFACE MODELS USING ELECTROPHYSIOLOGY MEASUREMENTS

(71) Applicant: ST. JUDE MEDICAL, CARDIOLOGY DIVISION, INC., St. Paul, MN (US)

(72) Inventors: Cyrille Casset, Saint Selve (FR); Jan O. Mangual-Soto, Rho (IT); Louis-Philippe Richer, Montreal (CA); Chunlan Jiang, Crystal, MN (US); Craig Markovitz, Mahtomedi, MN (US)

(21) Appl. No.: 17/432,686

(22) PCT Filed: Feb. 20, 2020
(86) PCT No.: PCT/US2020/018961

§ 371 Date: Aug. 20, 2021

Related U.S. Application Data
(60) Provisional application No. 62/809,153, filed on Feb. 22, 2019.

(51) Int. Cl. G06T 19/20; A61B 5/367; G06T 17/30

(52) U.S. Cl. CPC G06T 19/20; A61B 5/283; G06T 17/30; A61B 5/367

(57) ABSTRACT

Systems and methods for modifying a geometry surface model using electrophysiology (EP) measurements are provided. A system includes a device including at least one sensor configured to collect a set of location data points, and collect EP data at a measurement point. The system further includes a computer-based model construction system coupled to the device and configured to generate an original surface based on the set of location data points, the original surface including a plurality of corner points and a plurality of surface segments extending between the plurality of corner points, modify the original surface, based on the measurement point, to generate a modified surface, and map the EP data for the measurement point to the modified surface.

### CROSS REFERENCE TO RELATED APPLICATIONS

This application claims priority to provisional application Ser. No. 62/809,153, filed Feb. 22, 2019, which is incorporated herein in its entirety.

### FIELD OF THE DISCLOSURE

The present disclosure relates generally to generating geometry surface models for anatomical structures. In particular, in many aspects, the present disclosure relates to modifying geometry surface models using electrophysiology measurements.

### BACKGROUND

The human heart muscle routinely experiences electrical currents traversing its many surfaces and ventricles, including the endocardial surfaces. Just prior to each heart contraction, the heart muscle is said to “depolarize” and “repolarize,” as electrical currents spread across the heart and throughout the body. In a healthy heart, the surfaces and ventricles of the heart will experience an orderly progression of a depolarization wave. In an unhealthy heart, such as those experiencing atrial arrhythmia, including for example, ectopic atrial tachycardia, atrial fibrillation, and atrial flutter, the progression of the depolarization wave may not be so orderly. Arrhythmias may persist as a result of scar tissue or other obstacles to rapid and uniform depolarization. These obstacles may cause depolarization waves to repeat a circuit around some part of the heart. Atrial arrhythmia can create a variety of dangerous conditions, including irregular heart rates, loss of synchronous atrioventricular contractions, and stasis of blood flow, all of which can lead to a variety of ailments and even death.

Medical devices, such as, for example, mapping, electroporation, and/or electrophysiology catheters, are used in a variety of diagnostic and/or therapeutic medical procedures to treat such heart arrhythmias. Typically in a procedure, a catheter is manipulated through a patient's vasculature to a patient's heart, for example, and carries one or more electrodes that may be used for mapping, ablation, diagnosis, and/or to perform other functions.

At least some known model construction systems use a medical device to generate a geometry surface model of an anatomical structure. Further, using the medical device, electrophysiology measurements may be acquired for the anatomical structure. When mapping the electrophysiology measurements to the geometry surface model, however, there may be discrepancies between the geometry surface model and locations where the electrophysiology measurements are acquired.

### BRIEF SUMMARY OF THE DISCLOSURE

In one embodiment, the present disclosure is directed to a system for modifying a geometry surface model using electrophysiology (EP) measurements. The system includes a device including at least one sensor configured to collect a set of location data points, and collect EP data at a measurement point. The system further includes a computer-based model construction system coupled to the device and configured to generate an original surface based on the set of location data points, the original surface including a plurality of corner points and a plurality of surface segments extending between the plurality of corner points, modify the original surface, based on the measurement point, to generate a modified surface, and map the EP data for the measurement point to the modified surface.

In another embodiment, the present disclosure is directed to a computer-implemented method for modifying a geometry surface model using electrophysiology (EP) measurements. The method includes receiving a set of location data points, receiving EP data at a measurement point, generating an original surface based on the set of location data points, the original surface including a plurality of corner points and a plurality of surface segments extending between the plurality of corner points, modifying the original surface, based on the measurement point, to generate a modified surface, and mapping the EP data for the measurement point to the modified surface.

In yet another embodiment, the present disclosure is directed to a processing apparatus for modifying a geometry surface model using electrophysiology (EP) measurements. The processing apparatus is configured to receive a set of location data points, receive EP data at a measurement point, generate an original surface based on the set of location data points, the original surface including a plurality of corner points and a plurality of surface segments extending between the plurality of corner points, modify the original surface, based on the measurement point, to generate a modified surface, and map the EP data for the measurement point to the modified surface.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a diagrammatic view of a system for generating a multi-dimensional surface model of a geometric structure according to one embodiment.

FIG. 2 is a diagrammatic and schematic view of a model construction system of the system illustrated in FIG. 1.

FIG. 3 is a schematic view of a point cloud containing a collection of location data points.

FIGS. 4A-4D are schematic diagrams of exemplary dipole pairs of driven patch electrodes suitable for use in the model construction system illustrated in FIG. 2.

FIG. 5 is a flow diagram one embodiment of a method for modifying a geometry surface model using at least one measurement point where EP data is recorded.

FIG. 6A is a diagram illustrating a known method of mapping EP data to a surface.

FIGS. 6B and 6C are diagrams illustrating embodiments of modifying a surface based on at least one measurement point.

FIGS. 7A and 7B are diagrams illustrating another example of the embodiment shown in FIG. 6C.

FIGS. 8A and 8B are diagrams illustrating another embodiment of modifying a surface based on at least one measurement point.

FIGS. 9A and 9B are diagrams illustrating another embodiment of modifying a surface based on at least one measurement point.

FIGS. 10A-10C are diagrams illustrating another embodiment of modifying a surface based on at least one measurement point.

### DETAILED DESCRIPTION OF THE DISCLOSURE

The present disclosure provides systems and methods for dynamically modifying a geometry surface model based on at least one measurement point where EP data is recorded. Initially, a plurality of location data points are collected, and an initial surface including a plurality of corner points is generated based on the plurality of location data points. Further, EP data is recorded for at least one measurement point. The initial surface is dynamically modified based on the at least one measurement point. For example, the surface may be modified to include the at least one measurement point. As another example, at least one corner point of the plurality of corner points of the initial surface may be suppressed by replacing that corner point with the at least one measurement point (i.e., effectively moving that corner point to the at least one measurement point).

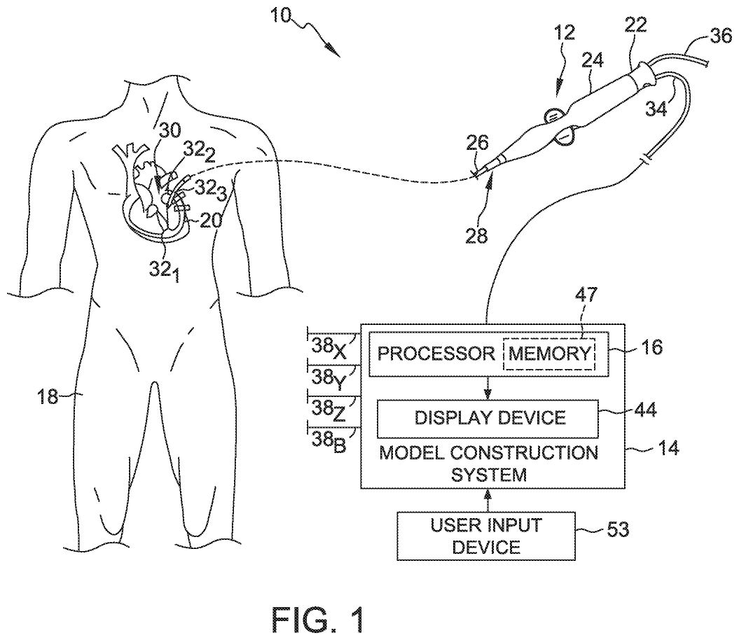

Referring now to the drawings wherein like reference numerals are used to identify identical components in the various views, FIG. 1 illustrates one exemplary embodiment of a system 10 for generating a multi-dimensional surface model of one or more geometric structures. As will be described below, in this embodiment, the model generated by system 10 is a three-dimensional model. It will be appreciated, however, that while the generation of a three-dimensional model is described below, the present disclosure is not meant to be so limited. Rather, in other embodiments, system 10 may be configured to generate multi-dimensional models other than in three-dimensions, and such embodiments remain within the spirit and scope of the present disclosure.

It should be further noted that while the following description focuses primarily on the use of system 10 in the generation of models of anatomic structures, and cardiac structures in particular, the present disclosure is not meant to be so limited. Rather, system 10, and the methods and techniques used thereby, may be applied to the generation of three-dimensional models of any number of geometric structures, including anatomic structures other than cardiac structures. However, for purposes of illustration and ease of description, the description below will be limited to the use of system 10 in the generation of three-dimensional models of cardiac structures.

With continued reference to FIG. 1, in this embodiment, the system 10 includes, among other components, a medical device and a model construction system 14. In this embodiment, the medical device is a catheter 12, and model construction system 14 includes, in part, a processing apparatus 16. Processing apparatus 16 may take the form of an electronic control unit, for example, that is configured to construct a three-dimensional model of structures within the heart using data collected by catheter 12

As illustrated in FIG. 1, catheter 12 is configured to be inserted into a patient's body 18, and more particularly, into the patient's heart 20. Catheter 12 may include a cable connector or interface 22, a handle 24, a shaft 26 having a proximal end 28 and a distal end 30 (as used herein, “proximal” refers to a direction toward the portion of the catheter 12 near the clinician, and “distal” refers to a direction away from the clinician and (generally) inside the body of a patient), and one or more sensors 32 (e.g., 32₁, 32₂, 32₃) mounted in or on shaft 26 of catheter 12. In this embodiment, sensors 32 are disposed at or near distal end 30 of shaft 26. Catheter 12 may further include other conventional components such as, for example and without limitation, a temperature sensor, additional sensors or electrodes, ablation elements (e.g., ablation tip electrodes for delivering RF ablative energy, high intensity focused ultrasound ablation elements, etc.), and corresponding conductors or leads.

Connector 22 provides mechanical, fluid, and electrical connection(s) for cables, such as, for example, cables 34, 36 extending to model construction system 14 and/or other components of system 10 (e.g., a visualization, navigation, and/or mapping system (if separate and distinct from model construction system 14), an ablation generator, irrigation source, etc.). Connector 22 is conventional in the art and is disposed at proximal end 28 of catheter 12, and handle 24 thereof, in particular.

Handle 24, which is disposed at proximal end 28 of shaft 26, provides a location for the clinician to hold catheter 12 and may further provide means for steering or guiding shaft 26 within body 18 of the patient. For example, handle 24 may include means to change the length of a steering wire extending through catheter 12 to distal end 30 of shaft 26 to steer shaft 26. Handle 24 is also conventional in the art and it will be understood that the construction of handle 24 may vary. In other embodiments, catheter 12 may be robotically driven or controlled. Accordingly, rather than a clinician manipulating a handle to steer or guide catheter 12 and shaft 26 thereof, in such an embodiments, a robot is used to manipulate catheter 12.

Shaft 26 is an elongate, tubular, flexible member configured for movement within body 18. Shaft 26 supports, for example and without limitation, sensors and/or electrodes mounted thereon, such as, for example, sensors 32, associated conductors, and possibly additional electronics used for signal processing and conditioning. Shaft 26 may also permit transport, delivery, and/or removal of fluids (including irrigation fluids, cryogenic ablation fluids, and bodily fluids), medicines, and/or surgical tools or instruments. Shaft 26 may be made from conventional materials such as polyurethane, and defines one or more lumens configured to house and/or transport electrical conductors, fluids, or surgical tools. Shaft 26 may be introduced into a blood vessel or other structure within the body 18 through a conventional introducer. Shaft 26 may then be steered or guided through body 18 to a desired location, such as heart 20, using means well known in the art.

Sensors 32 mounted in or on shaft 26 of catheter 12 may be provided for a variety of diagnostic and therapeutic purposes including, for example and without limitation, electrophysiological studies, pacing, cardiac mapping, and ablation. In this embodiment, one or more of sensors 32 are provided to perform a location or position sensing function. More particularly, and as will be described in greater detail below, one or more of sensors 32 are configured to be a positioning sensor(s) that provides information relating to the location (position and orientation) of catheter 12, and distal end 30 of shaft 26 thereof, in particular, at certain points in time. Accordingly, as catheter 12 is moved along a surface of a structure of interest of heart 20 and/or about the interior of the structure, sensor(s) 32 can be used to collect location data points that correspond to the surface of, and/or other locations within, the structure of interest. These location data points can then be used by, for example, model construction system 14, in the construction of a three-dimensional model of the structure of interest, which will be described in greater detail below. For purposes of clarity and illustration, the description below will discuss an embodiment wherein multiple sensors 32 of catheter 12 comprise positioning sensors. It will be appreciated, however, that in other embodiments, which remain within the spirit and scope of the present disclosure, catheter 12 may comprise both one or more positioning sensors as well as other sensors configured to perform other diagnostic and/or therapeutic functions.

As will also be described in greater detail below, in addition to performing the position sensing function described above, or in the alternative, one or more of sensors 32 may be configured to measure one or more EP parameters corresponding to the cardiac structure using techniques that are well known in the art. More particularly, as a sensor 32 that is configured to make such measurements is moved along the surface of the cardiac structure, sensor 32 is configured to make measurements of an EP parameter of interest and to communicate the measured value(s) of the parameter to the model construction system 14. The measured value(s) of the EP parameter can then be used by, for example, the model construction system 14, in the construction of an EP map of the cardiac structure on a geometry surface model of the cardiac structure.

While in an exemplary embodiment the position sensing function and EP parameter measurement functions may be performed by different sensors, for purposes of clarity and illustration, the description below will be limited to an embodiment wherein each of sensors 32 of catheter 12 is configured to perform the position sensing and measurement functions. It will be appreciated, however, that embodiments wherein different sensors are used to perform the different functions remain within the spirit and scope of the present disclosure.

As briefly described above, and as will be described in greater detail below, model construction system 14 is configured to construct a three-dimensional model (also referred to as a geometry surface model) of structures within the heart using, in part, location data collected by catheter 12. More particularly, processing apparatus 16 of model construction system 14 is configured to acquire location data points collected by sensor(s) 32 and to then use those location data points in the construction or generation of a model of the structure(s) to which the location data points correspond. In this embodiment, model construction system 14 acquires the location data points by functioning with sensors 32 to collect location data points. In other embodiments, however, model construction system 14 may simply acquire the location data points from sensors 32 or another component in system 10, such as, for example, a memory or other storage device that is part of model construction system 14 or accessible thereby, without affirmatively taking part in the collection of the location data points. Model construction system 14 is configured to construct a three-dimensional model based on some or all of the collected location data points. For purposes of illustration and clarity, the description below will be limited to an embodiment wherein model construction system 14 is configured to both construct the model and also acquire location data points by functioning with sensor(s) 32 in the collection of the location data points. It will be appreciated, however, that other embodiments wherein model construction system 14 only acquires location data points from sensor(s) 32 or another component of system 10 and then constructs a three-dimensional model based thereon remain within the spirit and scope of the present disclosure.

Further, in an exemplary embodiment, processing apparatus 16 is configured to use EP data/information collected by the catheter 12 to modify the three-dimensional model and generate a 3D map, as described in detail herein.

In some embodiments, system 10 may include an electrical field- and magnetic field-based system such as the ENSITE PRECISION™ system commercially available from Abbott Laboratories, and generally shown with reference to U.S. Pat. No. 7,263,397 entitled “Method and Apparatus for Catheter Navigation and Location and Mapping in the Heart”, the entire disclosure of which is incorporated herein by reference. In such embodiments, distal end 30 may include at least one magnetic field sensor—e.g., magnetic coils (not shown). If two or more magnetic field sensors are utilized, a full six-degree-of-freedom registration of magnetic and spatial coordinates could be accomplished without having to determine orthogonal coordinates by solving for a registration transformation from a variety of positions and orientations. Further benefits of such a configuration may include advanced dislodgement detection and deriving dynamic field scaling since they may be self-contained.

In other exemplary embodiments, system 10 may utilize systems other than electric field-based systems. For example, system 10 may include a magnetic field-based system such as the CARTO™ system commercially available from Biosense Webster, and as generally shown with reference to one or more of U.S. Pat. No. 6,498,944 entitled “Intrabody Measurement”; U.S. Pat. No. 6,788,967 entitled “Medical Diagnosis, Treatment and Imaging Systems”; and U.S. Pat. No. 6,690,963 entitled “System and Method for Determining the Location and Orientation of an Invasive Medical Instrument,” the disclosures of which are incorporated herein by reference in their entireties.

In yet another exemplary embodiment, system 10 may include a magnetic field-based system such as the GMPS system commercially available from MediGuide Ltd., and as generally shown with reference to one or more of U.S. Pat. No. 6,233,476 entitled “Medical Positioning System”; U.S. Pat. No. 7,197,354 entitled “System for Determining the Position and Orientation of a Catheter”; and U.S. Pat. No. 7,386,339 entitled “Medical Imaging and Navigation System,” the disclosures of which are incorporated herein by reference in their entireties.

In a further exemplary embodiment, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety. In yet still other exemplary embodiments, the subsystem 18 may comprise or be used in conjunction with other commonly available systems, such as, for example and without limitation, fluoroscopic, computed tomography (CT), and magnetic resonance imaging (MRI)-based systems.

As briefly described above, sensor(s) 32 of catheter 12 include positioning sensors. Sensor(s) 32 produce signals indicative of catheter location (position and/or orientation) information. Sensor(s) 32 may comprise one or more electrodes and/or one or more magnetic sensors configured to detect one or more characteristics of a low-strength magnetic field. For instance, in one exemplary embodiment, sensor(s) 32 may include magnetic coils disposed on or in shaft 26 of catheter 12.

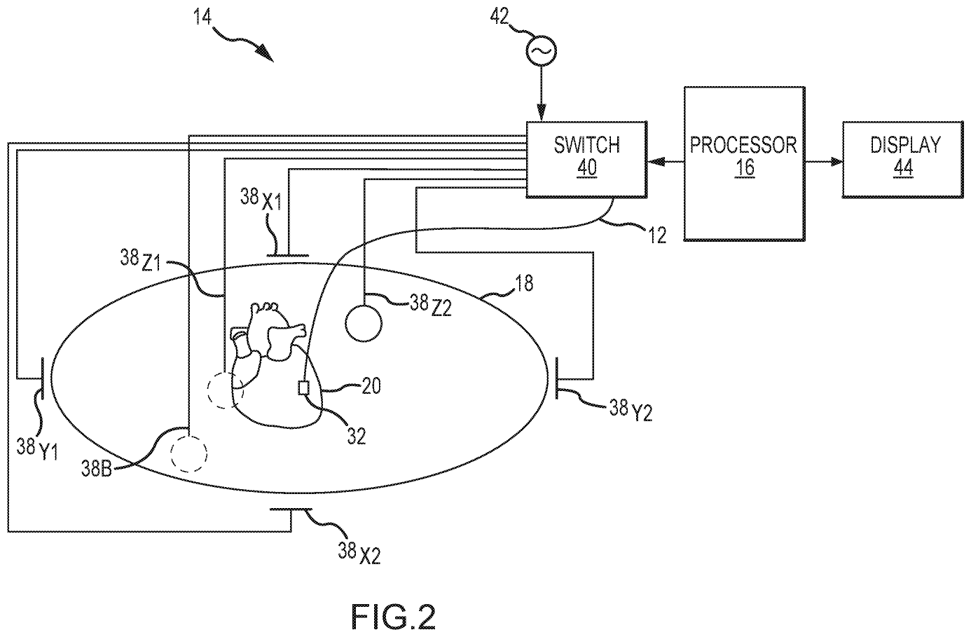

With reference to FIG. 2, in addition to the processing apparatus 16, model construction system 14 may include, among other possible components, a plurality of patch electrodes 38, a multiplex switch 40, a signal generator 42, and a display device 44. In other embodiments, some or all of these components are separate and distinct from model construction system 14 but are electrically connected to, and configured for communication with, model construction system 14.

Processing apparatus 16 may include a programmable microprocessor or microcontroller, or may include an application specific integrated circuit (ASIC). Processing apparatus 16 may include a central processing unit (CPU) and an input/output (I/O) interface through which the processing apparatus 16 may receive a plurality of input signals including, for example, signals generated by patch electrodes 38 and sensor(s) 32, and generate a plurality of output signals including, for example, those used to control and/or provide data to, for example, display device 44 and switch 40. Processing apparatus 16 may be configured to perform various functions, such as those described in greater detail above and below, with appropriate programming instructions or code (i.e., software). Accordingly, processing apparatus 16 is programmed with one or more computer programs encoded on a computer storage medium for performing the functionality described herein.

With the possible exception of patch electrode 38_{B} called a “belly patch,” patch electrodes 38 are provided to generate electrical signals used, for example, in determining the position and orientation of catheter 12. In one embodiment, patch electrodes 38 are placed orthogonally on the surface of body 18 and are used to create axes-specific electric fields within body 18. For instance, in one embodiment, patch electrodes 38_{X1}, 38_{X2} may be placed along a first (x) axis. Patch electrodes 38_{Y1}, 38_{Y2} may be placed along a second (y) axis, and patch electrodes 38_{Z1}, 38_{Z2} may be placed along a third (z) axis. Each of patch electrodes 38 may be coupled to multiplex switch 40. In this embodiment, processing apparatus 16 is configured, through appropriate software, to provide control signals to switch 40 to thereby sequentially couple pairs of electrodes 38 to signal generator 42. Excitation of each pair of electrodes 38 generates an electric field within body 18 and within an area of interest such as heart 20. Voltage levels at non-excited electrodes 38, which are referenced to belly patch 38_{B}, are filtered and converted and provided to processing apparatus 16 for use as reference values.

In this embodiment, sensor(s) 32 of catheter 12 are electrically coupled to processing apparatus 16 and are configured to serve a position sensing function. More particularly, sensor(s) 32 are placed within electric fields created in body 18 (e.g., within the heart) by exciting patch electrodes 38. For purposes of clarity and illustration only, the description below will be limited to an embodiment wherein a single sensor 32 is placed within electric fields. It will be appreciated, however, that in other embodiments that remain within the spirit and scope of the present disclosure, a plurality of sensors 32 can be placed within the electric fields and then positions and orientations of each sensor can be determined using the techniques described below.

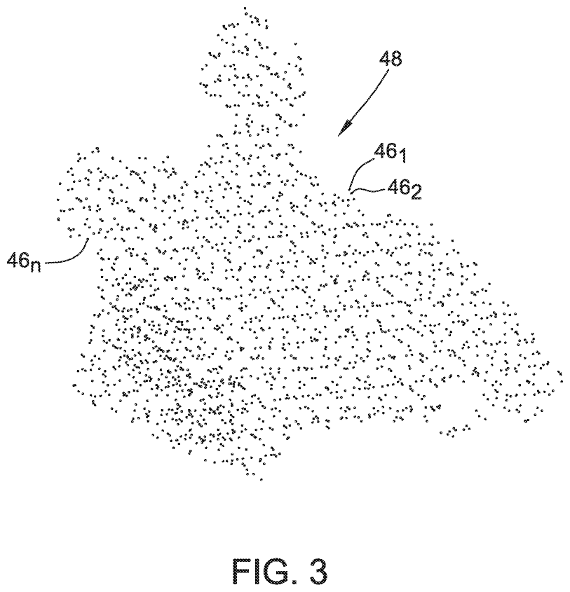

When disposed within the electric fields, sensor 32 experiences voltages that are dependent on the location between patch electrodes 38 and the position of sensor 32 relative to tissue. Voltage measurement comparisons made between sensor 32 and patch electrodes 38 can be used to determine the location of sensor 32 relative to the tissue. Accordingly, as catheter 12 is swept about or along a particular area or surface of interest, processing apparatus 16 receives signals (location information) from sensor 32 reflecting changes in voltage levels on sensor 32 and from the non-energized patch electrodes 38. Using various known algorithms, the processing apparatus 16 may then determine the location (position and orientation) of sensor 32 and record it as a location data point 46 (also referred to herein as “data point 46” and illustrated in FIG. 3) corresponding to a location of sensor 32, and therefore, a point on the surface or in the interior of the structure of interest being modeled, in a memory or storage device, such as memory 47, associated with or accessible by processing apparatus 16. In some embodiments, prior to recording the location as a location data point, the raw location data represented by the signals received by processing apparatus 16 may be corrected by processing apparatus 16 to account for respiration, cardiac activity, and other artifacts using known or hereafter developed techniques. Further, locations of other portions of catheter 12 may be interred from measurements at sensors 32, such as by interpolation or extrapolation, to generate further location data points 46. In any event, the collection of location data points 46 (46₁, 46₂, . . . , 46ₙ) taken over time results in the formation of a point cloud 48 (best shown in FIG. 3) stored in the memory or storage device.

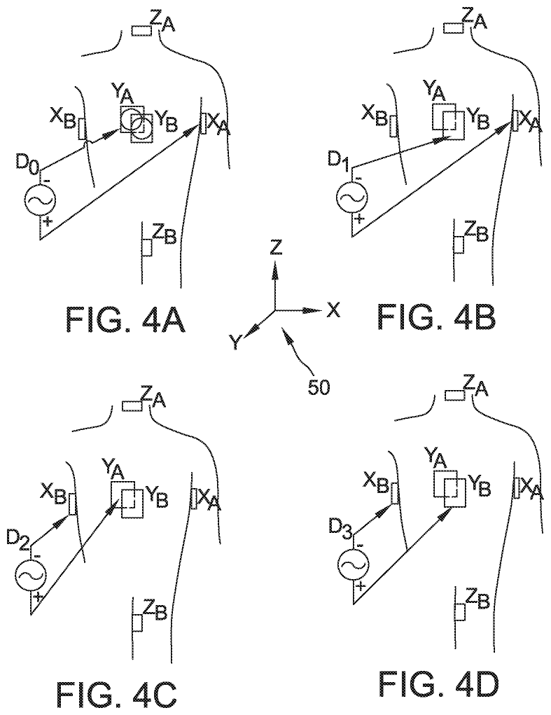

While the description above has thus far been generally with respect to an orthogonal arrangement of patch electrodes 38, the present disclosure is not meant to be so limited. Rather, in other embodiments, non-orthogonal arrangements may be used to determine the location coordinates of sensor 32. For example, and in general terms, FIGS. 4A-4D depict a plurality of exemplary non-orthogonal dipoles D₀, D₁, D₂, and D₃, set in a coordinate system 50. In FIGS. 4A-4D, the X-axis patch electrodes are designated X_{A} and X_{B}, the Y-axis patch electrodes are designated Y_{A} and Y_{B}, and the Z-axis patch electrodes are designated Z_{A} and Z_{B}. For any desired axis, the potentials measured across an intra-cardiac sensor, such as sensor 32, resulting from a predetermined set of drive (source sink) configurations may be combined algebraically to yield the same effective potential as would be obtained simply by driving a uniform current along the orthogonal axes. Any two of the patch electrodes 38_{X1}, 38_{X2}, 38_{Y1}, 38_{Y2}, 38_{Z1}, and 38_{Z2} (See FIG. 2) may be selected as a dipole source and drain with respect to a ground reference, e.g., belly patch 38_{B}, while the unexcited patch electrodes measure voltage with respect to the ground reference. Sensor 32 placed in heart 20 is also exposed to the field for a current pulse and is measured with respect to ground (e.g., belly patch 38_{B}).

In another exemplary embodiment, multiple patch electrodes 38 may be arranged linearly along a common axis. In such an embodiment, excitation of an electrode pair comprising one of patch electrodes 38 and an electrode mounted on catheter 12 generates an electric field. The non-excited patch electrodes 38 may then measure potentials that can be used to determine the position of sensor 32. Accordingly, in such an embodiment, the excitation of multiple electrode pairs comprising different patch electrodes 38 and the catheter-mounted electrode may be used to determine the position of sensor 32.

Data sets from each of patch electrodes 38 and the sensor 32 are all used to determine the location of sensor 32 within heart 20. After the voltage measurements are made, a different pair of patch electrodes 38 is excited by the current source and the voltage measurement process of the remaining patch electrodes 38 and sensor 32 takes place. Once the location of sensor 32 is determined, and as was described above, the location may be recorded as a data point 46 in the same manner described above. In some embodiments, prior to recording the location as a location data point, the raw location data represented by the signals received by processing apparatus 16 may be corrected by processing apparatus 16 to account for respiration, cardiac activity, and other artifacts using known or hereafter developed techniques. Accordingly, it will be appreciated that any number of techniques may be used to determine locations of sensor 32 and to, therefore, collect data points corresponding thereto, each of which remains within the spirit and scope of the present disclosure.

FIG. 3 is illustrative of the point cloud 48 including location data points 46₁, 46₂, . . . , 46ₙ corresponding to a particular structure of interest being modeled. It will be appreciated that in practice, the point cloud 48 would generally include hundreds to hundreds of thousands of data points 46. For purposes of illustration and ease of description, however, the description below will be limited to a point cloud having a limited number of location data points, such as, for example, point cloud 48 including location data points 46. It will be further appreciated that location data points 46 corresponding to different regions of the structure of interest may be collected. In such an embodiment, processing apparatus 16 may be configured to group data points 46 corresponding to the region of the structure of interest from which they were collected. As such, if there are two regions of the structure of interest, all of the location data points corresponding to a first region will be grouped together and form a first point cloud, while all of the data points corresponding to a second region will be likewise grouped together and form a second point cloud.

As described elsewhere herein, processing apparatus 16 is configured to acquire the location data points in a number of ways. In this embodiment, processing apparatus 16 acquires the location data points from sensor 32, which collects the location data points from the surface of the structure. In other embodiments, processing apparatus 16 acquires the sets of location data points by obtaining them from a memory or storage device that is part of or electrically connected to, and configured for communication with, processing apparatus 16. Accordingly, processing apparatus 16 may acquire the sets of location data points (and the location data points thereof) from one of any number of sources, each of which remain within the spirit and scope of the present disclosure. Using the respective sets of location data points 46, processing apparatus 16 is configured to generate surface models of each region of interest, as described herein.

Once one or more sets of location data points 46 are acquired, processing apparatus 16 is configured to generate a geometry surface model based on the location data points 46. To do so, and in general terms, processing apparatus 16 then uses the acquired one or more sets of location data points 46 to generate the geometry surface model using a surface reconstruction technique, such as an alpha-hull technique.

As was described above, in addition to being configured to obtain (e.g., construct or generate, or otherwise acquire) a geometry surface model of the cardiac structure, processing apparatus 16 is further configured to construct an EP map corresponding to the cardiac structure. Accordingly, upon the completion of, or simultaneous with, the construction of a geometry surface model corresponding to at least a portion of the cardiac structure, processing apparatus 16 is further configured to update the geometry surface model using EP information (also referred to herein as EP data), and thus, to construct an EP map of the cardiac structure. The EP information may relate to one or more EP parameters of the cardiac structure, such as, for example and without limitation, those described in great detail in U.S. Pat. No. 7,774,051 entitled “System and Method for Mapping Electrophysiology Information onto Complex Geometry,” the entire disclosure of which is incorporated herein by reference. To summarize, however, the EP parameters may include, for example, voltage measurements, peak-to-peak voltage measurements, electrograms, complex fractionated electrograms (CFE), and other time- and frequency-domain EP information. It will be appreciated by those of ordinary skill in the art that a single EP parameter or multiple EP parameters may be measured, and in certain embodiments, used to update the geometry surface model. Accordingly, embodiments wherein more than one EP parameter is measured remain within the spirit and scope of the present disclosure.

To construct the EP map, processing apparatus 16 is configured to first acquire EP information. More particularly, as sensor 32 (or sensors 32, in an embodiment wherein multiple sensors are used) is moved along the surface of the cardiac structure, sensor 32 is configured to make one or more measurements of an EP parameter of interest. In an exemplary embodiment, a measurement of the EP parameter is made in response to a user command. More particularly, in an exemplary embodiment, system 10 further comprises a user input device 53 (shown in FIG. 1), which may include a touch screen, a keyboard, a keypad, a button, a mouse, a graphical user interface having one or more user-selectable or user-inputtable fields, or some other user-controllable input device that is electrically connected to processing apparatus 16, through which a user may issue a command to make an EP parameter measurement. Alternatively, processing apparatus 16 may be configured to automatically make such a measurement upon detecting that an event, such as, for example, an activation, has occurred, or otherwise determines or detects that the information relating to the EP parameter being measured is reliable. In any event, by virtue of sensor 32 being electrically connected to processing apparatus 16, once a measurement is made or taken, an electrical signal produced by sensor 32 and representative of the measured value of the EP parameter is communicated to processing apparatus 16.

Regardless of how a measurement is triggered, each time a measurement is made, processing apparatus 16 is configured to determine the location (position and orientation) of sensor 32 that made the measurement. The location is recorded as measurement point in a memory or storage device associated with, or accessible by, processing apparatus 16, such as, for example, memory 47. Each measurement point is also associated and recorded with the measured EP parameter value that corresponds to that particular measurement point. In an exemplary embodiment, processing apparatus 16 is configured to determine the location of sensor 32, and therefore, the corresponding measurement point, in the same manner as that described above with respect to the determination of the location of sensor 32 and the corresponding location data point 46. As such, the description set forth above applies here with equal weight and will not be repeated, rather it is incorporated here by reference. The collection of measurement points taken over time results in a formation of a point cloud stored in a memory or storage device (such as memory 47), which, along with the EP information represented by the EP parameter values corresponding to each measurement point 46, may be used by the processing apparatus 16 to update the geometry surface model and construct the EP map.

As described above, system 10 is capable of providing accurate localization of catheter 12 during a procedure, providing EP maps visible on a geometry surface model, and ensuring relevant position information for therapy applications (e.g., ablation). To generate a geometry surface model, system 10 analyzes the volume that catheter 12 moves through within a cardiac chamber. Further, system 10 is capable of recording EP parameters at measurement points when one or more sensors 32 are in contact with a surface of the cardiac chamber. These EP parameters may be projected or otherwise mapped onto the geometry surface model to generate an EP map.

As explained above, when system 10 performs a measurement for an EP parameter, system 10 records the measured value for the EP parameter, as well as a measurement point specifying the location where the EP parameter was measured. Because the EP parameter is measured while the catheter 12 contacts the cardiac chamber surface, in many situations, the measurement point should generally lie on the surface of the generated geometry surface model. However, in some situations, the measurement point will lie ‘inside’ or ‘outside’ the modeled surface, even though the measurement point for the EP parameter and the location data points used to generate the geometry surface model were obtained accurately. For example, the actual anatomy of the patient may have moved or deformed over time between the acquisition of the location data points and the measurement point (e.g., due to wall motion during a heartbeat).

Regardless, if a measurement point does not lie on the generated surface, a user operating system 10 may doubt the accuracy of system 10. In at least some known systems, when the measurement point does not lie on the generated surface, the EP parameter acquired at the measurement point is projected onto the nearest portion of the generated surface. However, this may lead to inaccurate EP maps, particularly for complex geometries (e.g., veins, appendages, etc.). Further, this technique does not account for an orientation and/or contact force of catheter 12 during acquisition of the EP parameter.

Accordingly, as described below in detail, the systems and methods described herein dynamically modify an original surface of a geometry surface model based on at least one measurement point where EP data is recorded. More specifically, the original surface of the geometry surface model may be updated to include the measurement point, as described herein. For example, the geometry surface model may be modified over the course of a lengthy procedure, providing good localization of catheter 12 in the 3D volume.

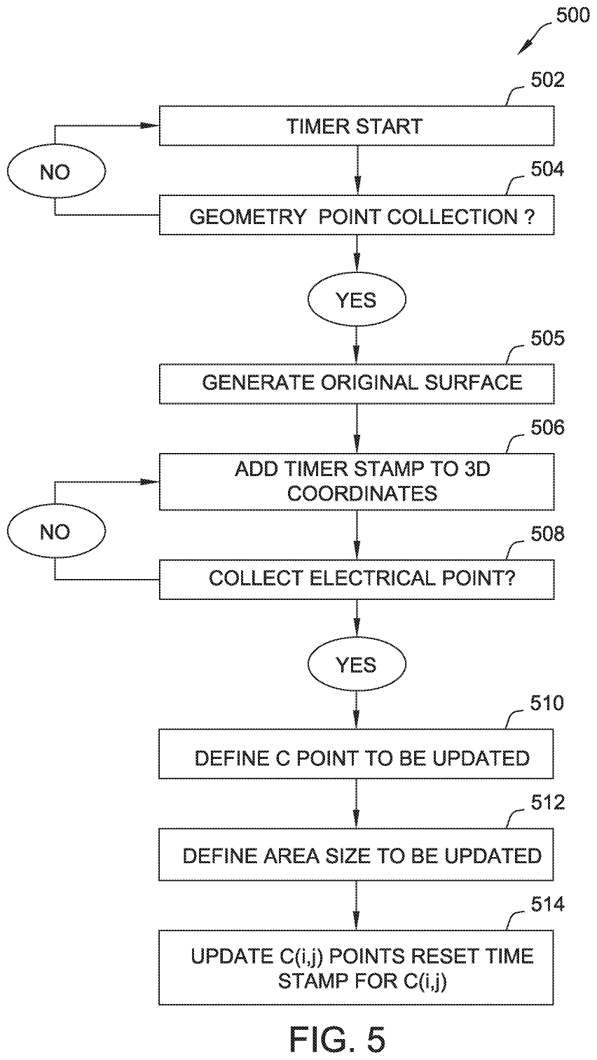

FIG. 5 is a flow diagram of one embodiment of a method 500 for modifying a geometry surface model using at least one measurement point where EP data is recorded. Method 500 may be implemented, for example, using system 10. At block 502, a timer is started. The timer may be implemented, for example, using processing apparatus 16. Flow proceeds to block 504, at which geometry points (e.g., location data points 46) are collected. If no geometry points are collected, flow returns to block 502. Otherwise, flow proceeds to block 505.

At block 505, an original surface is generated based on the collected geometry points. The original surface includes a plurality of corner points (which may correspond to the collected geometry points) and surface segments (also referred to as facets or subsurfaces) extending between the corner points, as described in detail below. Flow then proceeds to block 506.

In this embodiment, at block 506, using the timer, a time stamp is generated and stored for each corner point (indicating the time that the corner point was generated). Further, Flow then proceeds to block 508, at which electrical points (e.g., measurement points and associated EP data) are collected. If no electrical points are collected, flow returns to block 502. Otherwise, flow proceeds to block 510.

At block 510, at least one corner point to be updated is determined. Further, at block 512, a size of an area to be updated is determined. At block 514, the determined corner points are updated accordingly, and flow returns to block 506 to update the time stamp for any updated corner points. Examples and details regarding method 500 are described below.

The generation of a geometry surface model is described above. The geometry surface model defines a surface that is a spatial interface between the created volume (i.e., the volume traversed by distal end 30 of catheter 12) and the rest of the 3D space. The surface may be defined by a plurality of surface segments (also referred to as subsurfaces or facets) with a relatively simple geometry having a specific size, number or corners, or other fixed constraint. Depending on the chosen geometry, each surface segment has various contact lines (i.e., edges) shared with other surface segments. In one example, each surface segment is a quadrilateral that is defined by four location data points and shares edges with four other surface segments. Alternatively, the surface segments that make up the surface may have any suitable characteristics. Those of skill in the art will appreciate that various smoothing algorithms may also be applied to smooth the generated surface.

In some embodiments, once the geometry surface model is generated, EP data is acquired at various measurement points (See, e.g., block 508 in FIG. 5). In at least some known systems, for each measurement point, the EP data for that measurement point is projected onto the original surface at the subsurface closest to that measurement point. However, this has disadvantages, as described above. Accordingly, the systems and methods herein modify the original surface (i.e., by changing the shape of the original surface) based on the measurement points. Various methods of generating a modified surface based on measurement points are described herein. For clarity, the various methods are described relative to a two-dimensional surface. However, those of skill in the art will appreciate that the methods described herein are equally applicable to three-dimensional surfaces.

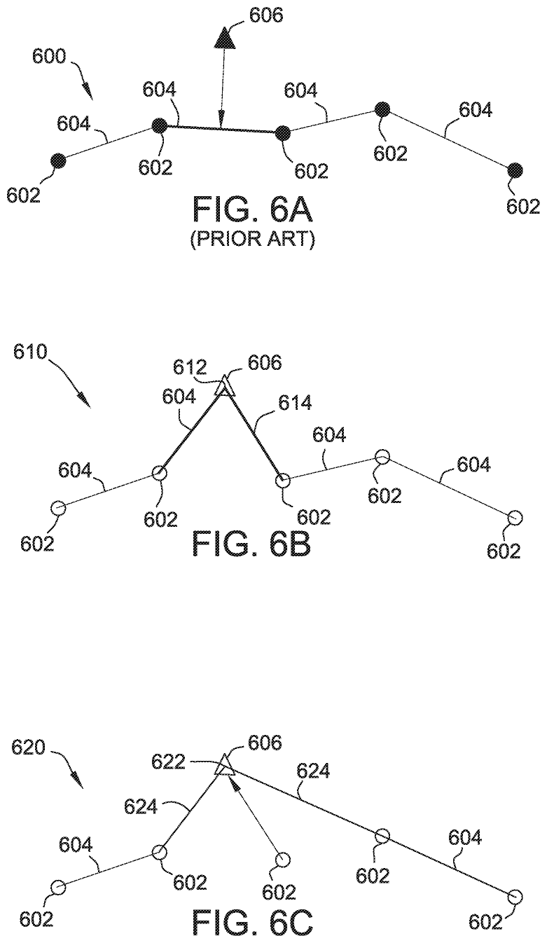

FIG. 6A is a diagram illustrating a known method of mapping EP data to a surface 600. FIGS. 6B and 6C are diagrams illustrating embodiments of modifying surface 600 based on at least one measurement point 606. Surface 600 includes five corner points 602 and four surface segments 604 extending between various pairs of the five corner points 602. Further, EP data is acquired at a measurement point 606.

FIG. 6A illustrates a known method of projecting EP data onto surface 600. Specifically, the EP data associated with measurement point 606 is projected onto the surface segment 604 that is closest to measurement point 606. Notably, this does not result in any modification (i.e., any change in shape) of surface 600.

In contrast, FIG. 6B illustrates one embodiment of a method of modifying surface 600 based on measurement point 606. Specifically, in the embodiment shown in FIG. 6B, the surface segment 604 that is closest to measurement point 606 is modified to include measurement point 606, without suppressing (i.e., removing) any of the original corner points 602. This results in a modified surface 610 that includes a new corner 612 and two new surface segments 614 (that replace one of the original surface segments 604).

FIG. 6C illustrates another embodiment of a method of modifying surface 600 based on measurement point 606. Specifically, in the embodiment shown in FIG. 6C, a modified surface 620 is generated by including measurement point 606 and suppressing (i.e., removing) the corner point 602 closest to measurement point 606. This results in modified surface 620 that includes a new corner 622 and two new surface segments 624 (that replace two of the original surface segments 604).

In the embodiments described herein, the Euclidian distance may be used to determine which corner point is closest to the measurement point. In at least some embodiments (e.g., the embodiment shown in FIG. 6C), the closest corner point will be suppressed and replaced by the measurement point.

Mathematically, the closest corner point may be calculated using the following:

$$C ( k , I ) / ∀ i , j : {D}_{e} ( P , {C}_{( k , l )} ) < {D}_{e} ( P , {C}_{( i , j )} )$$

$${D}_{e} ( P , {C}_{( i , j )} ) = \sqrt{{( {X}_{P} - {X}_{{C}_{( i , j )}} )}^{2} + {( {Y}_{P} - {Y}_{{C}_{( i , j )}} )}^{2} + {( {Z}_{P} - {Z}_{{C}_{( i , j )}} )}^{2}}$$

where D is the distance to be calculated, C(i,j) represents corner “i” of subsurface “j”, and P (X, Y, Z) are the coordinates of the measurement point. In other embodiments, parameters other than Euclidean distance may be used to determine the closest corner point.

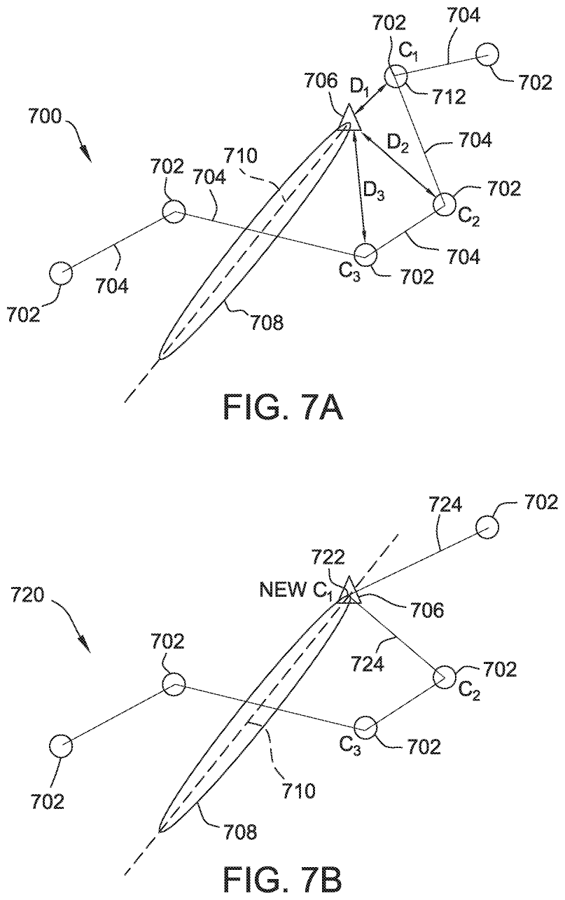

FIGS. 7A and 7B are diagrams illustrating another example of the embodiment shown in FIG. 6C. In this example, a surface 700 includes six corner points 702 and five surface segments 704 extending between various pairs of the six corner points 702. Further, EP data is acquired at a measurement point 706. FIGS. 7A and 7B also show a distal end 708 of a catheter (e.g., distal end 30 of catheter 12) used to acquire the EP data at measurement point 706. As shown in FIGS. 7A and 7B, distal end 708 is oriented along a catheter axis 710.

As described above, distances between corner points 702 and measurement point 706 are calculated to determine the corner point 702 closest to measurement point 706. As shown in FIG. 7A, a first corner point 712 (designated C1) is closest to measurement point 706 (i.e., a distance D1 is less than distances D2 and D3). Accordingly, to generate a modified surface 720 (shown in FIG. 7B), measurement point 706 is included and first corner point 712 is suppressed. This results in modified surface 720 that includes a new corner 722 and two new surface segments 724 (that replace two of the original surface segments 704).

In another embodiment, the surface is modified based at least in part on an orientation of the catheter when the EP data is acquired at the measurement point. In this embodiment, the distance calculation between corner points and the measurement point (represented mathematically above) may be modified to incorporate the catheter orientation (which may be determined by system 10, for example). Specifically, the calculated distance value may be multiplied by a catheter orientation factor that modifies the distance based on how closely the catheter axis aligns with a vector between a corner point and the measurement point for a given corner point. For example, the more the catheter axis aligns with the vector between the corner point and the measurement point, the more the modified distance is reduced for that corner point.

Mathematically, this may be represented as:

$${{D}_{e} ( P , {C}_{( i , j )} )}^{′} = ( 2 - \frac{( \vec{P , {C}_{( i , j )}} ) \cdot ( \vec{Cath} )}{‖ ( \vec{P , {C}_{( j , j )}} ) ‖ * ‖ ( \vec{Cath} ) ‖} ) * {D}_{e} ( P , {C}_{( i , j )} )$$

where (\vec{Cath}) is a vector aligned with the catheter axis and (\vec{P,C_{(i,j)}}) is a vector between a given corner point and the measurement point.

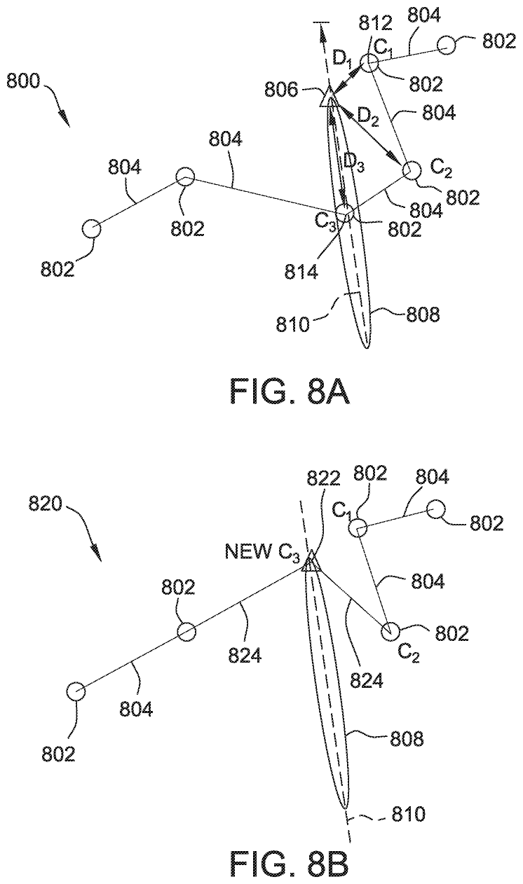

FIGS. 8A and 8B are diagrams illustrating this embodiment. A surface 800 includes six corner points 802 and five surface segments 804 extending between various pairs of the six corner points 802. Further, EP data is acquired at a measurement point 806. FIGS. 8A and 8B also show a distal end 808 of a catheter (e.g., distal end 30 of catheter 12) used to acquire the EP data at measurement point 806. As shown in FIGS. 8A and 8B, distal end 808 is oriented along a catheter axis 810.

Distances between corner points 802 and measurement point 806 are calculated to determine the corner point 802 “closest” to measurement point 806. As shown in FIG. 8A, based on Euclidean distance alone, a first corner point 812 (designated C1) is closest to measurement point 806 (i.e., a distance D1 is less than distances D2 and D3). However, when the calculated distances each are multiplied by a catheter orientation factor, as described above, a third corner point 814 (designated C3) has the shortest effective distance from measurement point 806 (due to catheter axis 810 and a vector between third corner point 814 and measurement point 806 being substantially aligned). Accordingly, to generate a modified surface 820 (shown in FIG. 8B), measurement point 806 is included and third corner point 814 is suppressed. This results in modified surface 820 that includes a new corner 822 and two new surface segments 824 (that replace two of the original surface segments 804).

In another embodiment, the surface is modified based at least in part on the catheter orientation and a contact force between the catheter and the cardiac tissue when the EP data is acquired at the measurement point. In this embodiment, the distance calculation between corner points and the measurement point (represented mathematically above) may be modified to incorporate the contact force (which may be determined by system 10, for example) as well as the catheter orientation. Specifically, the calculated distance value may be multiplied by the catheter orientation factor described above and a contact force factor that modifies the distance. For example, if the contact force is primarily along the catheter axis, the modified distance may be shorter than the actual distance. In contrast, if the contact force is primarily orthogonal to the catheter axis, the modified distance may be longer than the actual distance.

Mathematically, this may be represented as:

$${{D}_{e} ( P , {C}_{( i , j )} )}^{″} = {{D}_{e} ( P , {C}_{( i , j )} )}^{′} * ( 1 + ( 1 - \frac{( \vec{P , {C}_{( i , j )}} ) \cdot ( \vec{Cath} )}{‖ ( \vec{P , {C}_{( j , j )}} ) ‖ * ‖ ( \vec{Cath} ) ‖} ) * \frac{{F}_{a}}{{F}_{a} + {F}_{h}} + \frac{( \vec{P , {C}_{( i , j )}} ) \cdot ( \vec{Cath} )}{‖ ( \vec{P , {C}_{( j , j )}} ) ‖ * ‖ ( \vec{Cath} ) ‖} * \frac{{F}_{h}}{{F}_{a} + {F}_{h}} )$$

where Fₐ is a component of the contact force along the catheter axis, and Fₕ is a component of the contact force in a direction orthogonal to the catheter axis.

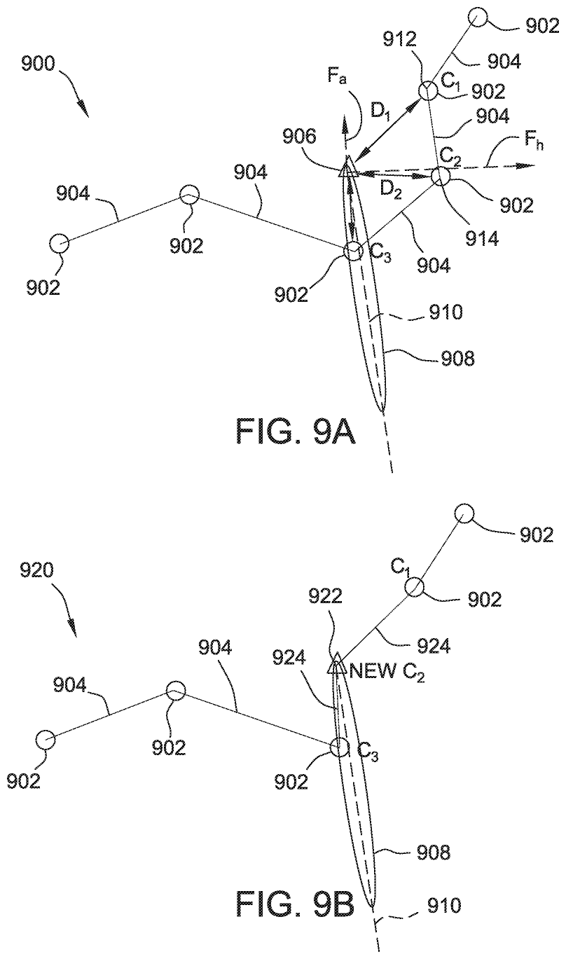

FIGS. 9A and 9B are diagrams illustrating this embodiment. A surface 900 includes six corner points 902 and five surface segments 904 extending between various pairs of the six corner points 902. Further, EP data is acquired at a measurement point 906. FIGS. 9A and 9B also show a distal end 908 of a catheter (e.g., distal end 30 of catheter 12) used to acquire the EP data at measurement point 906. As shown in FIGS. 9A and 9B, distal end 908 is oriented along a catheter axis 910.

Distances between corner points 902 and measurement point 906 are calculated to determine the corner point 902 “closest” to measurement point 906. As shown in FIG. 9A, based on Euclidean distance alone, a first corner point 912 (designated C1) may be closest to measurement point 906. However, when the calculated distances are each multiplied by a catheter orientation factor and a contact force factor, as described above, a second corner point 914 (designated C2) has the shortest effective distance from measurement point 906 (due to the contact force). Accordingly, to generate a modified surface 920 (shown in FIG. 9B), measurement point 906 is included and second corner point 914 is suppressed. This results in modified surface 920 that includes a new corner 922 and two new surface segments 924 (that replace two of the original surface segments 904).

Although the embodiment of FIGS. 9A and 9B includes modifying the surface based on both catheter orientation and contact force, those of skill in the art will appreciate that, in some embodiments, the surface may be modified based on contact force but not based on catheter orientation. This would be somewhat similar to the embodiment of FIGS. 8A and 8B, where the surface is modified based on catheter orientation but not based on contact force.

The embodiments described above in connection with FIGS. 6A-6C, 7A, 7B, 8A, 8B, 9A, and 9B, are various methods of implementing block 510 (shown in FIG. 5). Once system 10 (e.g., using processing apparatus 16) determines which corner point should be updated, system 10 determines (at block 512) the size of the area to be modified. Specifically, in one embodiment, the size of the area to be modified is defined as a number of corner points proximate the corner point (referred to herein as a primary corner point) that should also be updated.

For instance, in one example, a size of “0” indicates that only the primary corner point should be updated, a size of “1” indicates that all points directly connected to the primary corner point should also be updated, and a size of “2” includes that all corner points directly connected to corner points directly connected to the primary corner point should also be updated.

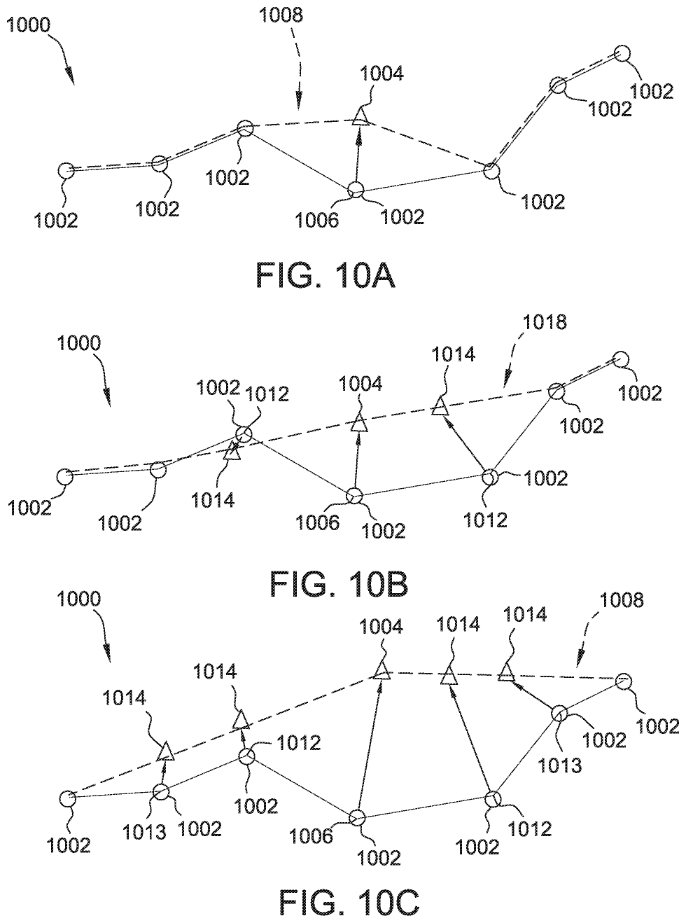

For example, referring to FIGS. 10A-10C, consider a surface 1000 including seven corner points 1002 and a measurement point 1004. Further, surface 1000 includes a primary corner point 1006 that is to be suppressed and replaced with measurement point 1004 (determined using, for example, the methods described above).

In FIG. 10A, the size is determined to be “0”. Accordingly, only primary corner point 1006 is updated (by being replaced with measurement point 1004 in a modified surface 1008).

In FIG. 10B, the size is determined to be “1”. Accordingly, primary corner point 1006 and all points directed connected to primary corner point 1006 (i.e., two additional points 1012) are updated. Specifically, in this example, primary corner point 1006 is replaced by measurement point 1004 in a modified surface 1018, and each additional point 1012 is replaced such that the updated additional point 1014 lies on a surface segment extending between measurement point 1004 and the nearest unmodified corner point 1002. Alternatively, primary corner point 1006 and additional points 1012 may be updated using any suitable methodology.

In FIG. 10C, the size is determined to be “2”. Accordingly, primary corner point 1006 and all points directed connected to primary corner point 1006 (i.e., two additional points 1012) and all points directly connected to additional points 1012 (i.e., two supplemental points 1013) are updated. Specifically, in this example, primary corner point 1006 is replaced by measurement point 1004 in a modified surface 1028, and each additional point 1012 and supplemental point 1013 is replaced such that the updated additional or supplemental point 1014 lies on a surface segment extending between measurement point 1004 and the nearest unmodified corner point 1002. Alternatively, primary corner point 1006, additional points 1012, and supplemental points 1013 may be updated using any suitable methodology.

In one embodiment, the size is calculated based on the time stamp associated with the primary corner point (as determined, for example, in block 506 of FIG. 5). For example, the size may be calculated using the following:

$$Size = Ent [ ( \frac{{T}_{{C}_{( i , j )}}}{{T}_{C}} ) * \frac{{{D}_{e} ( P , {C}_{( i , j )} )}^{″}}{{L}_{C}} ]$$

where T_{C(i,j)} is the time stamp for the primary corner point, T_{C} is a time constant (e.g., 60 seconds), L_{C} is a length constant (e.g., 2 millimeters(mm)) and Ent is a function that rounds to the closest integer number.

Using the above function, the size generally increases the longer ago that the time stamp for the primary corner point was generated. For example, if the time stamp was generated relatively recently (e.g., less than the time constant ago), the size may be “0”. On the other hand, if the time stamp was generated longer ago than the time constant, the size may be “1” or greater, depending on how long ago the time stamp was generated. In this embodiment, when a corner point is updated (i.e., moved), the time stamp for that corner point is reset.

After determining which corner points to update (e.g., at blocks 510 and 512 of FIG. 5), the corner points are updated accordingly (e.g., at block 514 of FIG. 5) to generate the modified surface. Those of skill in the art will appreciate that smoothing algorithms and/or other post-processing algorithms may be applied to the modified surface (similar to the initial surface). Further, in the embodiments described herein, map information (e.g., EP data) is associated with the corner points, not with 3D coordinates. Accordingly, as corner points are updated to new locations to generate the modified surface, any map information associated with a corner point is maintained when that corner point is updated (automatically incorporating the map information into the modified surface).

In at least some of the embodiments described herein, a corner point is updated by moving the corner point to the position of a measurement point (which is equivalent to suppressing the original corner point and adding the measurement point). However, in some embodiments, the corner point may be updated by moving the corner point only a predetermined portion of the distance towards the measurement point. The predetermined portion may be calculated, for example, based on a parameter (e.g., the time stamp of the corner point). For example, if the time stamp of the corner point was generated relatively long ago, the corner point may be moved the entire distance to the measurement point. However, if the time stamp of the corner point was generated relatively recently, the corner point may be moved only partially towards the sensor point. Those of skill in the art will appreciate that numerous methods for calculating the amount by which the corner point moves towards the measurement point may be used.

The systems and methods described herein are directed to dynamically modifying a geometry surface model based on at least one measurement point where EP data is recorded. Initially, a plurality of location data points are collected, and an initial surface including a plurality of corner points is generated based on the plurality of location data points. Further, EP data is recorded for at least one measurement point. The initial surface is dynamically modified based on the at least one measurement point. For example, the surface may be modified to include the at least one measurement point. As another example, at least one corner point of the plurality of corner points of the initial surface may be suppressed by replacing that corner point with the at least one measurement point (i.e., effectively moving that corner point to the at least one measurement point).

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A system for modifying a geometry surface model using electrophysiology (EP) measurements, the system comprising:
a device comprising at least one sensor configured to:
collect a set of location data points; and
collect EP data at a measurement point; and
a computer-based model construction system coupled to the device and configured to:
generate an original surface based on the set of location data points, the original surface including a plurality of corner points and a plurality of surface segments extending between the plurality of corner points;
modify the original surface, based on the measurement point, to generate a modified surface; and
map the EP data for the measurement point to the modified surface.

2. The system of claim 1, wherein to modify the original surface, the computer-based model construction system is configured to modify the original surface to include the measurement point.

3. The system of claim 1, wherein to modify the original surface, the computer-based model construction system is configured to:
for each corner of the plurality of corner points, calculate a distance between the corner point and the measurement point; and
modify the original surface based on the calculated distances.

4. The system of claim 3, wherein to modify the original surface based on the calculated distances, the computer-based model construction system is configured to:
determine a primary corner point that has the shortest calculated distance; and
modify the original surface by replacing the primary corner point with the measurement point on the modified surface.

5. The system of claim 3, wherein to modify the original surface based on the calculated distances, the computer-based model construction system is configured to:
adjust each of the calculated distances based on an orientation of the device when the EP data is collected; and
modify the original surface based on the adjusted distances.

6. The system of claim 3, wherein to modify the original surface based on the calculated distances, the computer-based model construction system is configured to:
adjust each of the calculated distances based on a contact force associated with the device when the EP data is collected; and
modify the original surface based on the adjusted distances.

7. The system of claim 1, wherein the computer-based model construction system is further configured to generate a time stamp for each of the plurality of corner points, and wherein to modify the original surface, the computer-based model construction system is further configured to modify the original surface based on the measurement point and the time stamps.

8. A computer-implemented method for modifying a geometry surface model using electrophysiology (EP) measurements, the method comprising:
receiving a set of location data points;
receiving EP data at a measurement point;
generating an original surface based on the set of location data points, the original surface including a plurality of corner points and a plurality of surface segments extending between the plurality of corner points;
modifying the original surface, based on the measurement point, to generate a modified surface; and
mapping the EP data for the measurement point to the modified surface.

9. The method of claim 8, wherein modifying the original surface comprises modifying the original surface to include the measurement point.

10. The method of claim 8, wherein modifying the original surface comprises:
for each corner of the plurality of corner points, calculating a distance between the corner point and the measurement point; and
modifying the original surface based on the calculated distances.

11. The method of claim 10, wherein modifying the original surface based on the calculated distances comprises:
determining a primary corner point that has the shortest calculated distance; and
modifying the original surface by replacing the primary corner point with the measurement point on the modified surface.

12. The method of claim 10, wherein modifying the original surface based on the calculated distances comprises:
adjusting each of the calculated distances based on an orientation of the device when the EP data is collected; and
modifying the original surface based on the adjusted distances.

13. The method of claim 10, wherein modifying the original surface based on the calculated distances comprises:
adjusting each of the calculated distances based on a contact force associated with the device when the EP data is collected; and
modifying the original surface based on the adjusted distances.

14. The method of claim 8, further comprising generating a time stamp for each of the plurality of corner points, and wherein modifying the original surface comprises modifying the original surface based on the measurement point and the time stamps.

15. A processing apparatus for modifying a geometry surface model using electrophysiology (EP) measurements, the processing apparatus configured to:
receive a set of location data points;
receive EP data at a measurement point;
generate an original surface based on the set of location data points, the original surface including a plurality of corner points and a plurality of surface segments extending between the plurality of corner points;
modify the original surface, based on the measurement point, to generate a modified surface; and
map the EP data for the measurement point to the modified surface.

16. The processing apparatus of claim 15, wherein to modify the original surface, the processing apparatus is configured to modify the original surface to include the measurement point.

17. The processing apparatus of claim 15, wherein to modify the original surface, the processing apparatus is configured to:
for each corner of the plurality of corner points, calculate a distance between the corner point and the measurement point; and
modify the original surface based on the calculated distances.

18. The processing apparatus of claim 17, wherein to modify the original surface based on the calculated distances, the processing apparatus is configured to:
determine a primary corner point that has the shortest calculated distance; and
modify the original surface by replacing the primary corner point with the measurement point on the modified surface.

19. The processing apparatus of claim 17, wherein to modify the original surface based on the calculated distances, the processing apparatus is configured to:
adjust each of the calculated distances based on an orientation of the device when the EP data is collected; and
modify the original surface based on the adjusted distances.

20. The processing apparatus of claim 17, wherein to modify the original surface based on the calculated distances, the processing apparatus is configured to:
adjust each of the calculated distances based on a contact force associated with the device when the EP data is collected; and
modify the original surface based on the adjusted distances.
